(19) United States
(12) Patent Application Publication
(10) Pub. No.: US 2005/0203580 A1
(43) Pub. Date: Sep. 15, 2005

(54) Method for multiple site, right ventricular pacing with improved left ventricular function

(76) Inventors: John K. Prentice, Boulder, CO (US); Mary Z. Fuka, Boulder, CO (US)

Correspondence Address: John K. Prentice, 505 Hapgood Street, Boulder, CO, 80302-6965, US

(21) Appl. No.: 10/837,309

(22) Filed: Apr. 30, 2004

Related U.S. Application Data
(63) Continuation-in-part of application No. 10/605,476, filed on Oct. 1, 2003.
Application No. 10/605,476 is a division of application No. 10/075,808, filed on Feb. 13, 2002, now Pat. No. 6,643,546.
(60) Provisional application No. 60/268,449, filed on Feb. 13, 2001.

(51) Int. Cl. A61N 1/362

(52) U.S. Cl. 607/9

(57) ABSTRACT

A method for treatment of congestive heart failure from the right side of the heart, by stimulating at numerous points along the right-ventricular septum to produce a fused line of stimulation upon breakthrough of wave fronts into the left ventricular septum and an LV action potential that simultaneously propagates toward the apex, base, and left free wall. Five electrodes, all in contact with the septum and spaced approximately 1.5 cm apart, produce a fused action potential in an average adult human within 10 ms of delivering simultaneous pacing pulses. Breakthrough of this fused region of stimulation will occur within 20 ms of delivering the pacing pulses. The most proximal electrode may be located in or near the right-ventricular apex. The most distal electrode may be located somewhere near the right-ventricular outflow tract, generally somewhere near the moderator band.

This application is a continuation-in-part of U.S. application Ser. No. 10/605,476, filed Oct. 1, 2003, which is a divisional application of U.S. application Ser. No. 10/075,808, filed Feb. 13, 2002, now U.S. Pat. No. 6,643,546 issued Nov. 4, 2003, which claimed the benefit of U.S. provisional application 60/298,449 filed Feb. 13, 2001.

### BACKGROUND OF THE INVENTION

This invention pertains to a method and apparatus for applying cardiac stimulation using multiple electrodes, and more particularly, to a method and apparatus for improving left ventricular function by multiple site pacing from the right ventricle.

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV), which then pumps it throughout the body. Four heart valves keep the blood flowing in the proper directions.

The electrical signal that drives this mechanical contraction starts in the sino-atrial node, a collection of specialized heart cells in the right atrium that automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles, causing them to contract and pump blood to the lungs and body. This electrical activity occurs approximately 72 times a minute in a normal individual and is called normal sinus rhythm.

The corresponding electrical signals identifying these events are usually referred to as the P, QRS (or R) and T waves or beats. More particularly, an atrial contraction is represented on an ECG (electrocardiogram) by a P wave, a ventricular contraction is represented by an R wave and a ventricular repolarization is represented by a T wave. The atrium also repolarizes but this event (the U wave) is masked by activity in the ventricle and consequently it is not observable on an ECG.

Congestive heart failure is a condition that causes many deaths annually. The condition is characterized by weakness, breathlessness, abdominal discomfort, and edema in the lungs and the lower portions of the body. These symptoms are associated with the inability of the heart to pump sufficient blood. Insufficiency may be associated with either the left ventricle, the right ventricle, or both. Cardiac output insufficiency may be caused by the failure of the heart to contract in an efficient way. If the physiologic conduction system has broken down or due to scar tissue associated with myocardial infarctions, the chambers of the heart may not contract in a coordinated or effective manner. In addition, dilated cardiomyopathy associated with heart failure often leads to a dysynchrony between the contraction of the left and right ventricles, to mitral regurgitation, and to paradoxical septal wall motion. All of these contribute to a reduction of cardiac output and to increased myocardial wall strain. In response to increased myocardial strain, the body produces myocardial stretch proteins and the heart expands. Reduction in myocardial strain reduces these stretch proteins and slows or reverses the progression of the dilated cardiomyopathy.

Paradoxical septal wall motion is also observed in patients who have undergone thoracic surgery. This may be due to neuro-hormonal imbalances, induced abnormal cardiac electrical conduction patterns, or other mechanisms that change the contraction pattern of the heart resulting in reduced cardiac output and increased myocardial strain.

In addition, artificial cardiac pacing using endocardial leads may diminish cardiac output, may compromise myocardial integrity, and may even, in some cases, precipitate heart failure. This is particularly true for pacing from the right ventricular apex. This may occur because current pacing modalities create a cardiac electrical and mechanical activation that significantly deviates from that of a healthy human in normal sinus rhythm with a functioning Purkinje system. This holds true both for hearts in which the normal sinus node activation or the Purkinje system is compromised, but which are otherwise healthy (for example, bradycardia, sick sinus syndrome, and bundle-branch block), as well as for hearts suffering from dilated cardiomyopathy or congestive heart failure. It is therefore of interest to develop pacing modalities that are as close as possible to the normal, Purkinje-generated activation sequence in a healthy heart-under normal sinus rhythm. Of particular importance is to generate an intraventricular coordination in the left ventricle (LV) that is as close to normal as possible.

Conventional pacemakers utilize a single or dual leads to apply pacing pulses. The dual (bipolar) lead typically includes a tip and a ring electrode. The lead is inserted in such a manner that the tip is imbedded into the cardiac muscle. A pacing pulse is then applied between the tip and the ring electrodes, thereby causing the cardiac muscle to contract. If a single unipolar electrode lead is used, the electric pulse is applied between the tip electrode and another electrode outside the heart, for example, the housing of the pacemaker. Bradycardia pacing therapy has usually been delivered through a pacing electrode implanted near the ventricular apex, that is, near the bottom of the heart. This location has been preferred not for physiologic reasons, but because most lead designs favor implantation at this site. A lead entering the right ventricle from the right atrium tends to extend into the lower apex of the ventricle where an active fixation apparatus, such as a helical corkscrew, may be used to secure the lead to the heart wall. The physician is thus limited to a single site for applying treatment. Bradycardia pacing therapy can be improved by delivering the stimulating pulse to a more efficient right-ventricular location than the ventricular apex. Studies have indicated that the abnormal contraction that results from apical pacing has long-term deleterious effects. Studies using conventional pacing leads implanted in alternative locations in the right ventricle (for example, the right-ventricular outflow tract or pacing both the right-ventricular apex and right-ventricular outflow tract) have shown clinical improvements, but the long-term reliability of conventional pacing leads in these alternative locations is questionable and lead placement is difficult. In addition, outcomes in alternate site pacing have been observed to be very sensitive to the location of the pacing site. This makes it difficult or impossible to achieve clinically reliable outcomes.

Such controlled stimulation may result in more efficient cardiac contraction, thereby reducing the overall demand on the heart, allowing the body to alleviate the symptoms associated with inefficient blood flow.

Cardiac pacing as practiced today involves venous access, endocardial pacing from the right atrium, venous access endocardial pacing from the right ventricle, venous access epicardial pacing of the left ventricle from the coronary venous system, epicardial pacing of the left ventricle from the outside of the heart, and various other combinations of these modalities. All of these modalities lead to activation sequence irregularities and consequence diminished cardiac function. However, the degree of LV intraventricular dysynchrony induced by these pacing modalities varies.

Right ventricular apical pacing, with or without atrial coordination, is known to produce the worst LV intraventricular synchrony of any current pacing modality. Right ventricular outflow tract pacing has been shown to improve cardiac performance, presumably by improving LV intraventricular synchrony. Left ventricular and bi-ventricular pacing (otherwise known as cardiac resynchronization therapy) have been shown to produce cardiac activation sequences that are better than right apical pacing and that lead to correspondingly better cardiac performance. Recent research on right-septal bifocal pacing has shown clinical outcomes similar to bi-ventricular pacing, presumably by also improving LV intraventricular synchrony. Left ventricular apical endocardial pacing has been shown to produce the best LV intraventricular synchrony of all these pacing modalities, but it is not practical in a clinical setting due to the dangers of leaving a permanent pacing lead in the left ventricle. Right atrial pacing in patients with a healthy AV node, bundle-branch, and Purkinje system has been shown to produce a near normal activation sequence, as does His-bundle pacing. However, these therapies are limited to the relatively few pacing patients with fully functioning conduction systems. In addition, His-bundle pacing is difficult to produce reliably in a clinical setting.

One useful quantitative measure of the degree of intraventricular electrical synchronization is the LV activation coordination factor, defined as:

$$\frac{{\int }_{LV} ⅆ V \vert {τ}_{normal} ( \vec{r} ) - {τ}_{paced} ( \vec{r} ) \vert}{V} + 1$$

where τₙₒᵣₘₐₗ(\vec{r}) is the time at which the action potential reaches a point \vec{r} in the left ventricle during normal activation, ι_{paced}(\vec{r}) is the time at which the action potential reaches a point \vec{r} in the left ventricle during paced activation, V is the volume of the left ventricle (including the intraventricular septum), and the volume integral is taken over the myocardium of the left ventricle, including the intraventricular septum. A value of 1 for this activation factor means that an intraventricular synchrony has been generated that is identical to that of a normal, healthy heart in sinus rhythm. Larger values indicate a poorer degree of LV intraventricular synchrony.

FIG. 11 shows the LV activation coordination factor for various pacing modalities based on a realistic computer simulation. RVA refers to right ventricular endocardial apical pacing, LV refers to left ventricular epicardial pacing, RVOT refers to endocardial right ventricular outflow tract pacing, RVOT/RVA refers to endocardial right-ventricular septal pacing simultaneously from the RVA and RVOT, BiV refers to bi-ventricular pacing, and LVA refers to endocardial left ventricular apical pacing. A decreasing LV activation coordination factor should correlate with improved cardiac hemodynamics. This is in agreement with in vivo data collected in otherwise healthy dogs with induced left bundle branch block. Intraventricular activation synchrony improves left to right in this FIG. 11. However, there is still substantial deviation from a normal activation pattern.

At one time it was thought that the key to improving cardiac performance in pacing was to narrow the width of the QRS complex, producing a better synchronization between the right and left ventricles, i.e., inter-ventricular synchronization. This was the initial goal of cardiac resynchronization therapy. However, it is now well documented that shortening the QRS complex to achieve improved inter-ventricular synchronization is not generally associated with improved LV intra-cardiac synchronization. Although U.S. Pat. No. 5,174,289 discloses the use of multiple electrodes on a single right-ventricular lead to deliver simultaneous pacing pulses to the right-ventricular septum for generating a narrow QRS complex, it does not recognize the importance of producing a uniform wave front at the left ventricular septal wall or inducing an activation pattern that is as similar as possible to a physiologically normal pattern in space and time throughout the LV.

### OBJECTIVES AND SUMMARY OF THE INVENTION

In view of the above disadvantages of the prior art, it is an objective of the present invention to provide an implantable cardiac stimulation system, such as a pacemaker, in which three or more electrodes are positioned in a chamber of the heart and an optimum electrode or electrodes are selected for stimulating the left ventricular septal wall.

It is also an object of the invention to provide an implantable cardiac stimulation system, such as a pacemaker, in which three or more electrodes are positioned in a chamber of the heart and all of the electrodes are used to simultaneously stimulate the left ventricular septal wall.

It is also an object of the invention to place a plurality of electrodes along the right ventricular septal wall surface of a heart, select a set of said electrodes such that stimulation wave fronts from the selected electrodes will stimulate substantially all of the left ventricular septal wall surface.

It is also an object of the invention to place a plurality of electrodes along the right ventricular septal wall surface of a heart and use all of the electrodes to generate simultaneous stimulation wave fronts in order to stimulate substantially all of the left ventricular septal wall surface.

Another object is to produce a stimulating pulse at each electrode in a set of electrodes on the right ventricular wall surface at substantially the same time (or precisely simultaneously) to produce a fused line (or region) of stimulation along the left ventricular septal wall surface.

It is also an object of the invention to place a set of electrodes on the right ventricular wall surface, generate an elliptical wave front from each electrode such that areas are formed on the left ventricular wall surface where the wave fronts emerge during a pre-selected time.

A further object is to select electrodes on the right ventricular septal wall such that adjacent areas of the wave fronts emerging on the left ventricular septal wall impinge on each other.

Another object is to stimulate all the electrodes simultaneously on the right ventricular septal wall to initiate a circumferentially propagating LV epicardial and endocardial action potential.

Another object of the invention is to produce a fused line (or region) of stimulation extending from about the left ventricular apex to about the mitral valve.

Another object of the invention is to produce a fused line (or region) of stimulation extending from about the left ventricular apex to about the left ventricular outflow tract.

Another object of the invention is to produce an initially disjoint but large region of stimulation covering a substantial percentage of the LV septal wall extending from about the left ventricular apex to about the mitral valve.

Another object of the invention is to produce an initially disjoint but large region of stimulation covering a substantial percentage of the LV septal wall extending from about the left ventricular apex to about the left ventricular outflow tract.

Another object of the invention is to produce an initially disjoint but large region of stimulation covering a substantial percentage of the RV septal wall extending from about the right ventricular apex to about the tricuspid valve and some portion of the right ventricular free wall.

Another object of the invention is to produce an initially disjoint but large region of stimulation covering a substantial percentage of the RV septal wall extending from about the right ventricular apex to about the right ventricular outflow tract and some portion of the right ventricular free wall.

Briefly, the subject invention pertains to an implantable cardiac stimulation system having a cardiac stimulator having electronic circuitry for the stimulation and a multi-electrode lead attached to the stimulator and inserted into one or more body cavities. (The term cardiac stimulator will be used herein to cover pacemakers as well as other cardiac devices such as internal cardioversion devices and defibrillators.) The lead is inserted into the cardiac cavity into a predetermined position. Alternatively the lead may be positioned in the veins, or it may be positioned externally of the heart. Since the lead has many electrodes, an appropriate subset of electrodes may be selected for stimulation. Alternately, all the electrodes may be used in concert for simultaneous stimulation.

This disclosure discusses stimulating the heart at numerous points along the right-ventricular septum to produce a fused line (or region) of stimulation upon breakthrough of the action potential into the left-ventricular septum and simultaneously an action potential propagating through the LV myocardium from the RV septum toward the apex, base, and left free wall of the LV.

The goal is to more closely mimic the Purkinje septal activation sequence in the left ventricle that would occur in a normal heart. Applications of this therapy include the treatment of Bradycardia (or any other disease requiring artificial cardiac pacing) and congestive heart failure. This therapy is also of value in heart transplants where artificial pacing is necessary and for the treatment of irregular cardiac contraction following thoracic surgery. The goal is to generate improved cardiac performance and also minimize myocardial wall strain in order to prevent, slow, or reverse the progression of heart failure.

The cardiac conduction system consists of the sin sino-atrial (SA) node, intra-atrial conduction fibers, atrio-ventricular (AV) node, His bundle, bundle branches, and the Purkinje system. Impulses from the SA node are conducted through this system to the ventricles. The right bundle branch lies on the right side of the inter-ventricular septum and does not branch until it reaches the right ventricular apex where it anastamoses with the Purkinje fibers to conduct the impulses to the right ventricle. The left bundle branch lies on the left side of the septum and almost immediately divides into two main divisions, the anterior superior and the posterior inferior division. Each subdivides into a number of fascicles. The anterior superior division supplies the anterior and superior wall, and the posterior inferior division supplies the posterior interior wall of the left ventricle. The two fascicles anastomose at the periphery and carry impulses to the respective area supplied through the Purkinje fibers. There is probably a third septal division also, but it has not been recognized electrocardiographically. In humans, total ventricular activation lasts between 62 and 80 ms, corresponding to a QRS duration of 70 to 80 ms.

In the left ventricle, unlike the right ventricle, the left bundle branch almost immediately splits into Purkinje fibers located along the length of the left ventricular septum from the LV outflow tract to the LV apex. They then continue along the apex of the left ventricle and part way up the LV free wall. When an action potential is conducted through the Purkinje fibers in the left ventricle, it causes a rapid activation of the left ventricular septum followed almost immediately by activation of the apex and lower regions of the free wall.

When the LV conduction system is diseased, for example by left-bundle branch block or a myocardial infarction, normal Purkinje activation is not possible. In these cases, activation occurs predominantly via slow conduction of an action potential from the right ventricle, through the inter-ventricular septum, and into the LV. Breakthrough of the action potential from the RV to LV occurs at one or two sites on the LV septum. Alternately, the activation may be through only a portion of the LV Purkinje system. In any of these cases, the resulting activation of the LV typically exhibits a high degree of LV intraventricular dyssynchrony. Similarly, most kinds of cardiac pacing, for example pacing for the treatment of Bradycardia or congestive heart failure, typically overrides the Purkinje system and generates LV intraventricular dysynchrony.

The method of this invention aims to use pacing to mimic the activation of the left ventricular septum by the Purkinje system. This involves stimulating at numerous points along the right-ventricular septum to produce a fused line (or region) of stimulation upon breakthrough of wave fronts into the left ventricular septum and an LV action potential that simultaneously propagates toward the apex, base, and left free wall, called herein Purkinje Replacement Therapy or Physiological Resynchronization Therapy or “PRT”. Computer simulations of PRT indicate it produces the best LV intraventricular synchronization of any pacing modality that has heretofore been proposed.

An interesting consequence of PRT is to produce a shortened QRS complex. This is due to improving activation synchronicity over the entire heart, not just the left ventricle. However, it should be emphasized that narrowing the QRS complex is not the goal of PRT, rather it is a mere side effect, and that mere narrowing of the QRS has not been shown to correlate clinically with improved cardiac function.

Delivering PRT involves stimulating the right ventricular septum substantially simultaneously from roughly the apex to the outflow tract. Since simultaneous activation of the RV septum is desired, this therapy should be deliverable with a standard pacemaker. The right-ventricular lead or leads will connect to the ventricular channel. However, conventional lead technology will not be adequate for delivering PRT.

There are several lead technologies that may be suitable for PRT. One method would be to lay a single long coiled electrode, something like a defibrillator lead coil, along the septum. This type may present difficulties in anchoring the lead, achieving proper septal contact, and excessive battery drain due to the low impedance of such a coil. Further, in practice it is sufficient to stimulate the entire RV septum simultaneously at numerous locations sufficient to generate a fused line (or region) of stimulation when the action potential breaks through the septum into the left ventricle. This could be done using multiple standard ventricular pacing leads or a single lead that bifurcates into multiple leads at the distal end (Y leads). Endocardial RV patch electrodes could also be used to deliver simultaneous stimulation from an array of electrodes in the RV.

The leads described in U.S. Pat. No. 6,480,747 (incorporated herein by reference) consist of insulated conductors that terminate at a wound coil electrode. However, PRT is not dependent on any specific structure or technology for delivering stimulating pulses to the RV septum or on specific ways of constructing leads or electrodes. The electrodes could be wound coils as in the U.S. Pat. No. 6,480,747 lead or more conventional ring electrodes. Any anchoring technique can be used so long as a sufficient number of electrodes contact the septal wall (it is not necessary that all electrodes contact the wall so long as a sufficient number do and that they more or less evenly stimulate the RV septum). It is also not required that the conductors be insulated from each other since PRT may involve stimulating all electrodes substantially simultaneously. Insulated conductors may allow stimulating at different electrodes at slightly different times. It is preferred that the electrodes have a small surface area, for example between 1 mm² and 9 mm². The small electrodes will generate lower pacing thresholds and will minimize battery drain when placed in parallel. PRT could also be delivered by non-conventional means, for example, by the use of ultrasound from multiple piezoelectric crystals positioned on the RV septum.

The right-ventricular septum is approximately 5 or 6 cm long in adult humans. Sufficient electrodes, or more generally stimulation sites, are required along the RV septum so that when the action potential from each electrode reaches the LV side of the septum, the individual waves will have fused within some time, generally 1 to 10 ms. Alternately, the individual waves can be disjoint upon breakthrough into the LV septum, but upon breakthrough they should uniformly cover at least 50% of the activatable tissue on the LV septum. Because the action potential conduction velocity through (perpendicular to) the septum is smaller than along (parallel to) it, the action potential generated by each electrode will spread in an ellipsoid through the septal myocardium with the longer semi-major axis parallel to the septum and the shorter semi-minor axis transverse (perpendicular) to the septum. Geometrical analysis indicates that five electrodes, all in contact with the septum and spaced approximately 1.5 cm apart in a line, are sufficient to produce a fused action potential interior to the septal myocardium in an average adult human within 10 ms of delivering simultaneous pacing pulses with these electrodes. Breakthrough of this fused region of stimulation will occur within 20 ms of delivering the pacing pulses. The most proximal electrode is assumed to be located in or near the right-ventricular apex. The most distal electrode is assumed to be located somewhere near the right-ventricular outflow tract, generally somewhere near the moderator band. Other electrode geometries are possible, for example an array of patch electrodes. No matter what the geometry, it is necessary that the electrodes be separated from each other by no more than 1.5 cm in order to fuse together within 10 ms.

More than five electrodes in a line oriented along the long axis of the RV septum, or a patch of electrodes deploying at least five electrodes along the RV septal long axis and some number of electrodes more anterior and posterior, will generate a fused action potential on the LV septum in less time. A preferred embodiment for a line of electrodes on the RV septum along the long axis is 9 or 10 electrodes spaced 6 or 7 mm apart. This allows for the possibility that some of the electrodes may not be in good contact with the myocardium. PRT will be delivered so long as at least four or five of these electrodes, spread approximately evenly apart, are in contact with the septum.

In addition to stimulating at a minimum of five locations along the RV septal long axis, additional electrodes can be located on the right ventricular free wall to improve contraction of the RV. This is not necessary for optimizing LV activation, but may be of value in optimizing RV activation and is a way of treating RV heart failure.

PRT can also be delivered in conjunction with a LV lead for biventricular pacing. The LV lead electrode(s) could be fired simultaneously with the RV electrodes or within a pre-selected interval to optimize the overall contraction of the LV.

These and other objects and features of the invention will be apparent from the following detailed description, together with the accompanying drawings.

### BRIEF DESCRIPTION OF THE DRAWINGS

FIG. 1 shows a diagrammatic front view of a patient with a cardiac stimulation system, including a programmer used to program the cardiac stimulator.

FIG. 2 shows a block diagram of the cardiac stimulator or FIG. 1.

FIG. 3 is a block diagram of a portion of the circuits of FIG. 2.

FIG. 4 is a second embodiment of the circuit portion of FIG. 3.

FIG. 5 is a block diagram of another portion of the circuits of FIG. 2.

FIG. 6 is a second embodiment of the other circuit portion of FIG. 5.

FIG. 7 is a block diagram of an adapter for connecting a multi-electrode lead to an IS-1 connector.

FIG. 8 is a view of a multi-electrode lead implanted in a heart.

FIG. 9 is a cross section of the multi-electrode lead of FIG. 8.

FIG. 10 is a computer model result showing the spread of the action potential for a heart in normal sinus rhythm and with the method of this invention.

FIG. 11 shows the LV activation coordination factor for various pacing modalities based on a computer simulation.

### DETAILED DESCRIPTION OF THE INVENTION

The subject invention pertains to an implantable cardiac stimulation system 10 including a cardiac stimulator 12 with various electronic circuits, and a multi-electrode lead 14 attached to the stimulator 12, as shown. The lead 14 has a distal end 16 disposed in the right ventricle 18 of heart 20. The system 10 is adapted to deliver therapy in the form of electrical pulses. The cardiac stimulator 12 contains electronic components common to current cardiac stimulators such as a battery, microprocessor control circuit, ROM, RAM, an oscillator, reed switch and antenna for communication, output circuits, and sense circuits. These components are well known to those of skill in the art. It is believed that a standard pacemaker, capable of unipolar or bipolar pacing and equipped with what is known as an “IS-1” connector could be used to deliver the therapy described herein, provided a sufficient number of electrodes are deployed. These electrodes may be of small size, to reduce power drain during stimulation. The cardiac stimulator 12 may also have a plurality of sensing and stimulating circuits, as will be explained below, but such a configuration is not necessary to deliver the therapy.

Cardiac Stimulator

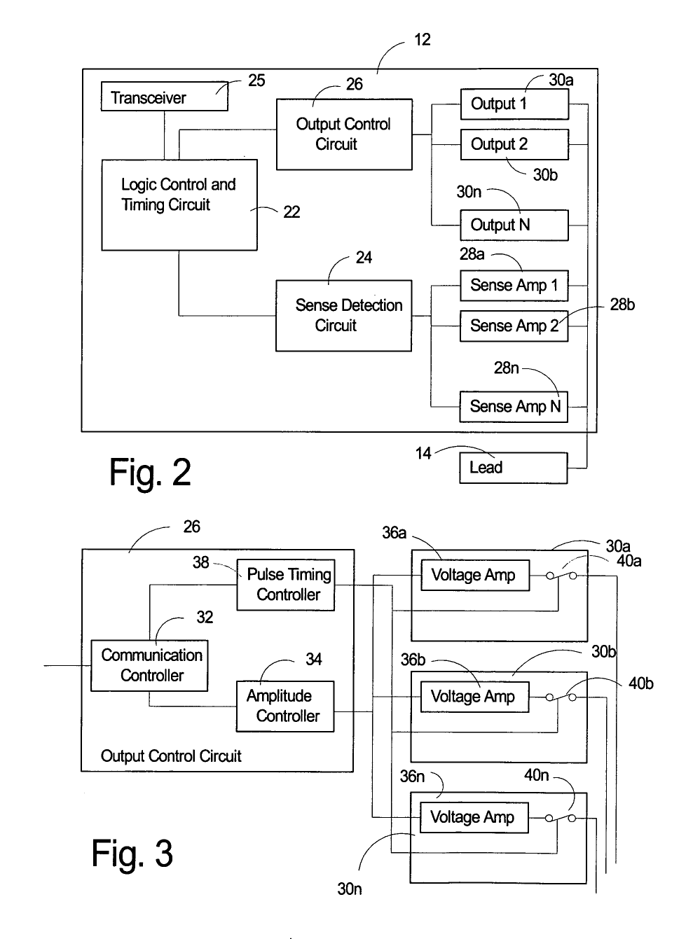

FIG. 2 illustrates important elements of the cardiac stimulator 12 in block diagram. The cardiac stimulator 12 comprises a logic control and timing circuit 22, which may include a microprocessor and memory, but which could also be implemented in a specialized circuit. The logic control and timing circuit 22 receives input from a sense detection circuit 24 and issues control instructions to an output control circuit 26. To accommodate the many electrodes used in the apparatus, multiple sense amplifiers 28a, 28b . . . 28n are provided, each in electrical communication with an electrode through the lead 14 and with the sense detection circuit 24. Similarly, the output control circuit 26 is electrically connected to a plurality of output circuits 30a, 30b . . . 30n. The output circuits 30a, 30b . . . 30n produce stimulating pulses or high frequency, non-simulating signals at electrodes in the heart through the lead 14. The logic control and timing circuit 22 may operate in accordance with a program stored into memory. The programming in memory is received through a transceiver 25 (for instance from programmer 100). As part of this programming, the electrodes designated for stimulation, as described below, are stored in memory. During its operation, the microprocessor of the logic control and timing circuit 22 sets the output control circuit 26 and the sense detection circuit 24 in accordance with the appropriate electrode designations. Thereafter, the sense detection circuit 24 senses intrinsic activity and other signals within the heart 20 and provides corresponding indication signals to the microprocessor. The logic control and timing circuit 22 then issues appropriate commands to the output control circuit 26. The output control circuit 26 generates appropriate stimulation pulses. These pulses are steered to the designated electrode or electrodes.

Output Circuits

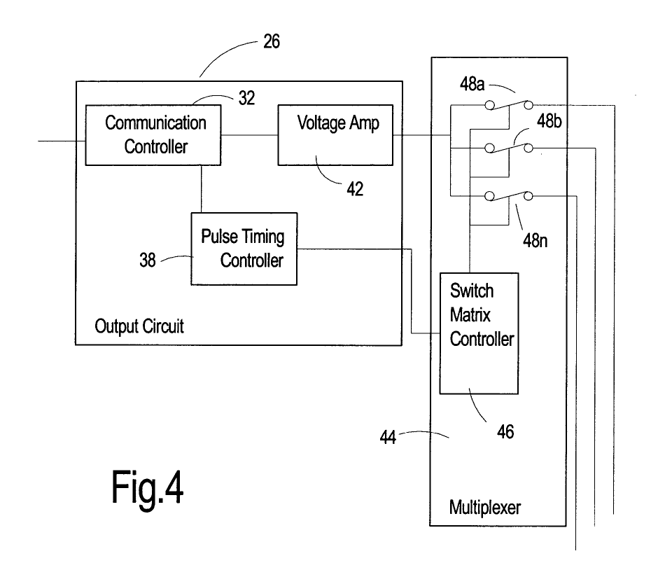

FIGS. 3 and 4 show two embodiments of output control circuit 26 and output circuits 30a, 30b . . . 30n. The embodiment of FIG. 3 comprises a communications controller that receives control signals from the logic control and timing circuit 22 (FIG. 2). Output of the communications controller 32 is sent to an amplitude controller 34 that controls the voltages produced by a plurality of voltage amplifiers 36a, 36b . . . 36n. In parallel, the communications controller 32 also regulates a pulse timing controller 38. Signals from the pulse timing controller 38 close and open switches 40a, 40b . . . 40n, thereby delivering stimulation pulses or high frequency signals to the heart through electrodes on the lead 14.

The embodiment of FIG. 4 also uses a communication controller 32 and pulse timing controller 38, but the amplitude controller 34 and plurality of voltage amplifiers 36a, 36b . . . 36n are replaced by a single voltage amplifier 42. To achieve the same effect of multiple pulses to selected electrodes, the signals from the pulse timing controller are sent to a multiplexer 44, comprising a switch matrix controller 46 and a plurality of switches 48a, 48b. . . 48n. The switches 48a, 48b . . . 48n must be opened and closed in a synchronized manner. It may be necessary to open all switches before and after closing a selected switch. Thus the embodiment of FIG. 4 gains simplicity and energy efficiency by minimizing the number of voltage amplifiers, but sacrifices flexibility in potential output patterns.

Sense Circuits

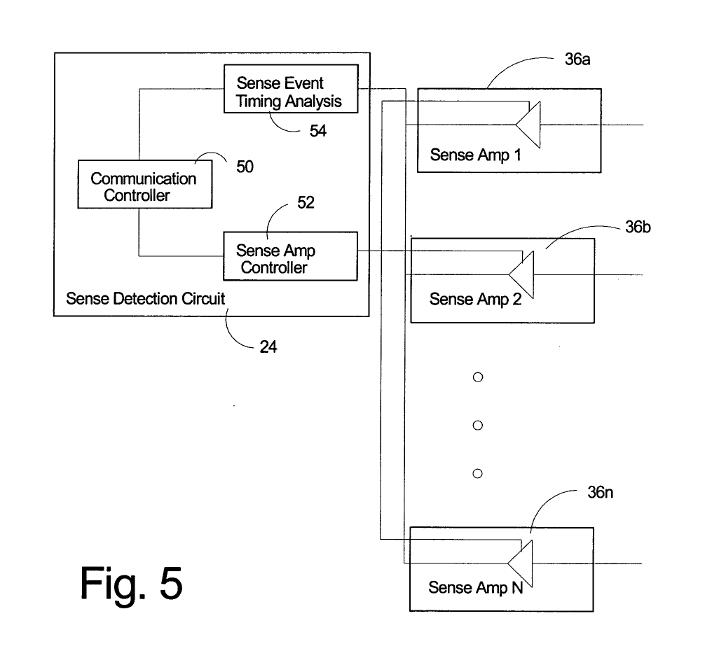

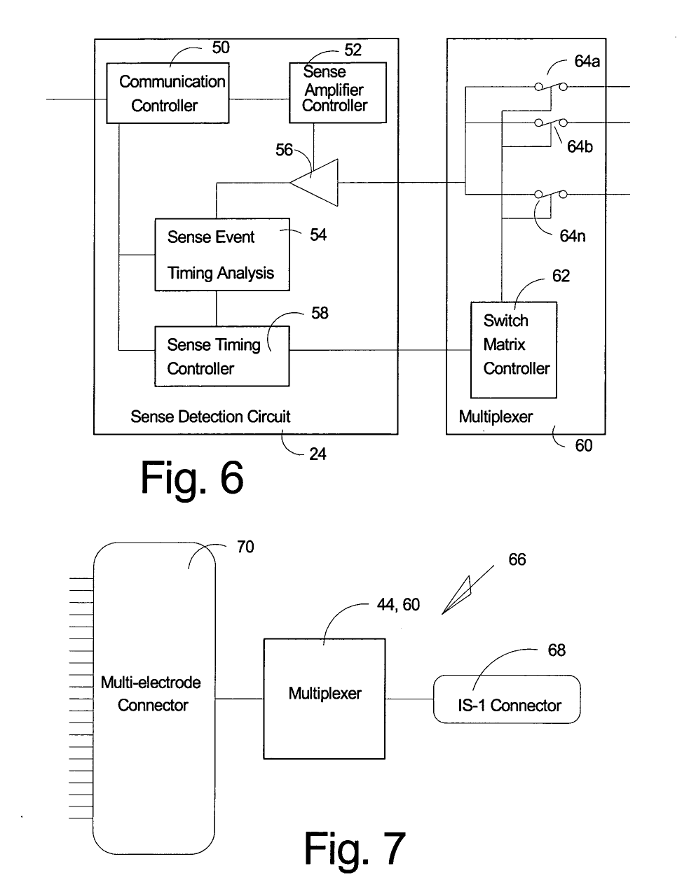

A variety of apparatus may also be used to sense signals from multiple electrodes through the sense detection circuit 24. A first embodiment is illustrated in FIG. 5. In the embodiment of FIG. 5, a communication controller 50 in the sense detection circuit 24 communicates with the logic control and timing circuit 22 (FIG. 2). The communication controller 50 is in electrical communication with a sense amp controller 52 and a sense event timing analysis unit 54. The sense amp controller 52 regulates amplification levels on the sense amps 36a, 36b . . . 36n such that significant signals are detected and noise is rejected. Each amplifier has independent sensitivity (gain) and filter characteristics. The sense event timing analysis unit 54 receives output from the sense amps 36a, 36b . . . 36n and collects that information into a description of a moving wave front. Both intervals between sensed events and the sequence of channels or electrodes are used to describe the wave front. The description of the wave front is communicated to the logic control and timing circuit 22 for use in determining the appropriate therapy. A second embodiment, illustrated in FIG. 6, employs a multiplexer in a manner similar to the second embodiment of the output control circuit, described in connection with FIG. 4, above. In this second embodiment of the sense detection circuit 24, the sense amp controller 52 controls a single amplifier 56. The sense event timing analysis unit 54 analyses the output of the single amplifier 56 and produces the description of the moving wave front. A sense timing controller 58, in electrical communication with both the communication controller 50 and the sense event timing analysis unit 54, controls a multiplexer 60 through a switch matrix controller 62. The switch matrix controller 62 opens and closes a plurality of switches 64a, 64b . . . 64n, selectively connecting the electrodes of the lead 14 to the sense amplifier 56. As explained above, replacing multiple dedicated sense amplifiers 36a, 36b . . . 36n with a single amplifier 56 exchanges flexibility and simplified control for energy efficiency.

The multiplexers 44, 60 of the embodiments of the output control circuit of FIG. 4 and of the sense detection circuit of FIG. 6 may be combined externally to the cardiac stimulator 12 in an alternative configuration, illustrated in part in FIG. 7. FIG. 7 shows an adapter 66 for a connecting a multi-electrode lead to a cardiac stimulator having an IS-1 connector in the header of the stimulator 12. IS-1 connectors are well known and many physicians are familiar with their operation and use. For the adapter 68 a male IS-1 connector 68 is connected to the multiplexers 44, 60 in an independent package. The multiplexers are connected either directly to the lead 14 or indirectly through a multi-electrode connector 70. Dual chamber pacemakers having two IS-1 connectors in a single header are well known. In cardiac stimulators 12 according to the present invention using IS-1 connectors rather than a specialized multi-electrode connector, a first IS-1 connector might be used to carry both the voltage from the voltage amp 42 and signals from the pulse timing circuit 38 and a second IS-1 connector might be used to carry both the signals to the sense amplifier 56 and the control signals from the sense timing controller 58. Alternatively, one IS-1 connector might be dedicated to the control signals from the sense timing controller 58 and the pulse timing circuit 38 while another IS-1 connector might be dedicated to the signals delivered to and received from the heart, that is, to pulses from the voltage amp 42 and to sensed events.

Multi-Electrode Lead

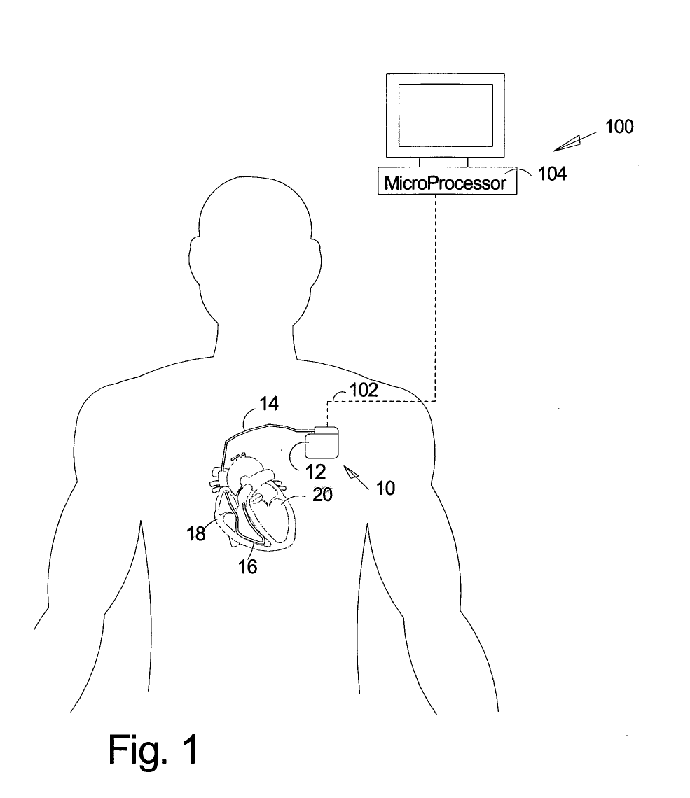

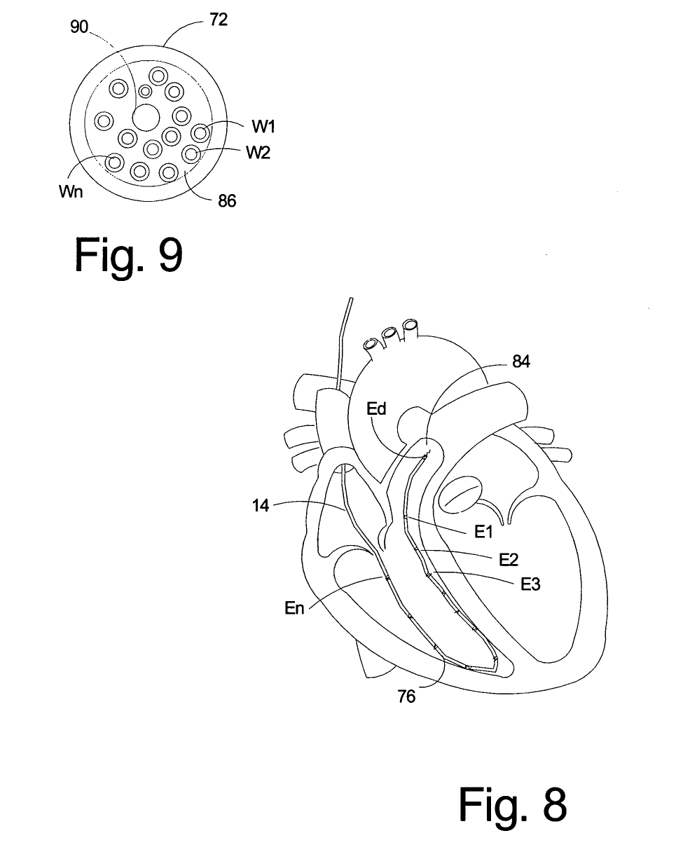

Details of the multi-electrode lead 14 are shown in FIG. 8 and FIG. 9. In one embodiment, the lead 14 includes an external biocompatible polymer tube 72 having a straight portion 74 and a shaped portion 76. The tube may be made of polyurethane or other similar materials that may be thermally shaped so that the shaped portion 76 retains any desired configuration. In FIGS. 1 and 8, the shaped portion 76 is shown as a folded lead that places electrodes along the ventricular septum and up into the right ventricular outflow tract. Although other shapes may be selected, this embodiment may be particularly useful where the applied therapy seeks to stiffen the septum, as further described below.

Attached to tube 72 of the lead 14 of any configuration, there are provided a plurality of electrodes E1, E2, E3, E4, E5, . . . En. Preferably electrodes E1 . . . En are formed of coils of bare wire or cable wound about the tube 72. Each electrode is connected to corresponding wires W1, W2, W3 . . . Wn which extend through the length of tube 72. Wires W1, W2, W3 . . . Wn may be insulated, but need not be for certain aspects of this invention. The electrode 14 and its method of manufacture are disclosed in application Ser. No. 09/761,333 filed Jan. 16, 2001, now U.S. Pat. No. 6,480,747 and incorporated herein by reference. Preferably, the end of tube 72 and the ends of wires W1, W2, W3, etc. are coupled to a connector for attaching the lead 14 to the cardiac stimulator 12. In addition to spiral coil or ring electrodes E1 . . . En, a distal tip electrode Ed may also be provided. The distal tip electrode Ed may also have an active fixation mechanism, for example a helical screw 84 or tines, to secure the lead to the interior wall of the heart.

The tube 72 can be formed with a longitudinal cavity 86, as shown in the cross sectional view of FIG. 9. Cavity 86 holds the wires W1, W2, W3 etc. The lead 14 could be straightened by inserting a substantially straight stylet 90 into cavity 86. The stylet 90 is also flexible but is less flexible than the lead 14 so that as it is inserted into the cavity 86, it forces the tube 72 to straighten. The lead 14 is then inserted into the heart or into a vein near the heart. After implantation of the lead 14, the stylet 90 is withdrawn and the lead 14 flexes back and takes a predetermined configuration.

The multi-electrode lead can also be used in an embodiment with electrodes E1 through En are shorted together at the proximal connector. Another embodiment would short all but one of the electrodes together, the remaining independent electrode being used as a common anode. In either embodiment, it would also be permissible for the shorted electrodes to be uninsulated.

A programmer 100 may be used to program the cardiac stimulator 12, usually by electromagnetic signals. The programmer 100 comprises a microprocessor 104 for performing various functions in connection with programming the cardiac stimulator.

This disclosure discusses a new approach to pacing called herein “Purkinje Replacement Therapy”, “Physiological Resynchronization Therapy”, or “PRT”. This involves stimulating at numerous points along the right-ventricular septum to produce a fused line of stimulation upon breakthrough of the action potential into the left-ventricular septum. The goal is to more closely mimic the Purkinje septal activation sequence in the left ventricle that would occur in a normal heart. Applications of this therapy include the treatment of Bradycardia and congestive heart failure. This therapy is also of value in heart transplants where artificial pacing is necessary. The goal is to generate improved cardiac performance and minimize myocardial wall strain in order to prevent or slow the progression of heart failure.

When the LV conduction system is diseased, for example by left-bundle branch block or a myocardial infarction, normal Purkinje activation is not possible. In these cases, activation occurs via slow conduction of an action potential from the right ventricle, through the inter-ventricular septum, and into the LV. Breakthrough of the action potential from the RV to LV occurs at one or two sites on the LV septum. The resulting activation of the LV typically exhibits a high degree of LV intraventricular dyssynchrony. Similarly, any kind of right-ventricular pacing, for example right-ventricular pacing for the treatment of Bradycardia or congestive heart failure, typically overrides the Purkinje system and generates this same kind of RV to LV action potential.

Purkinje Replacement Therapy, also known as Physiological Resynchronization Therapy, (PRT in either case) aims to use pacing to mimic the activation of the left ventricular septum by the Purkinje system. This involves stimulating at numerous points along the right-ventricular septum to produce a fused line (or region) of stimulation upon breakthrough into the left-ventricular septum. Computer simulations of this pacing modality indicate it produces the best LV intraventricular synchronization of any pacing modality that has heretofore been proposed while also producing an excellent RV intraventricular synchronization and good RV/LV inter-ventricular synchrony. Calculated results for the LV activation coordination factor for PRT (labeled PRT) are shown in FIG. 3, along with the results for all other current pacing modalities.

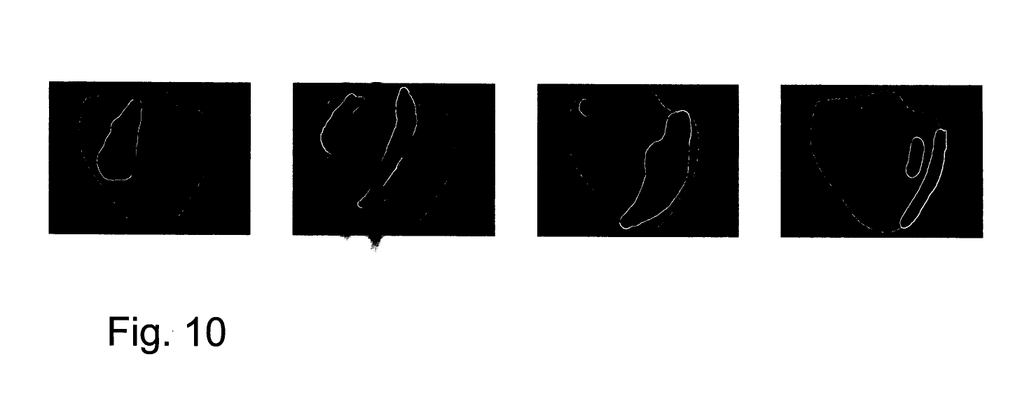

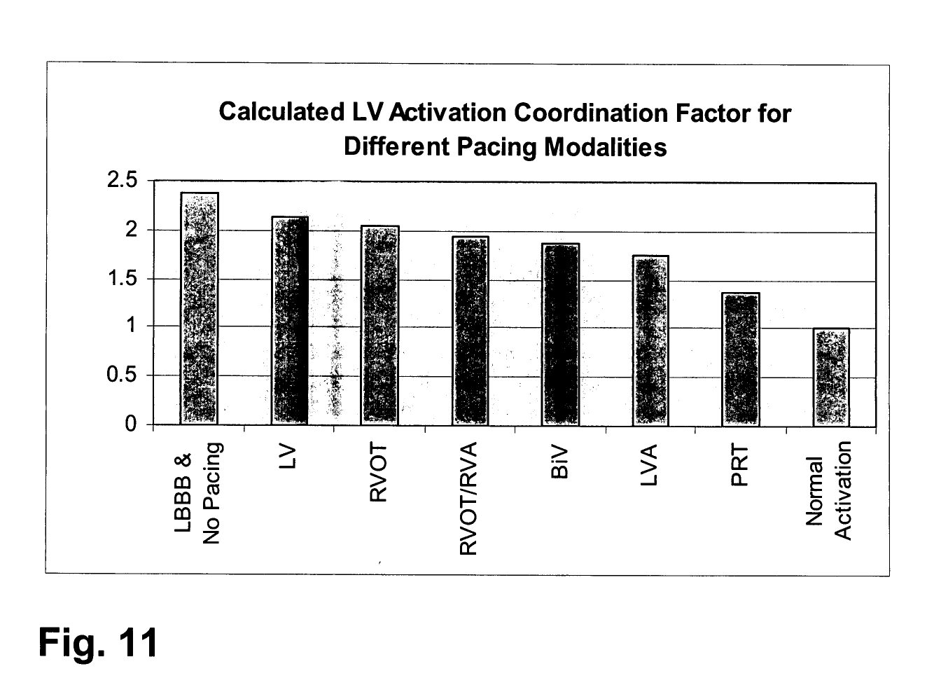

FIG. 10 shows computer model results showing the spread of the action potential for a healthy heart in normal sinus rhythm and with PRT. While the PRT paced heart does not have an activation sequence that is identical to a normal heart, it does generate a “Purkinje like” large region of activation of the LV septum and initiates a LV action potential that propagates around the circumference of the heart and along the base and apex to produce a near physiologically normal activation pattern. The cycle numbers in the figure represent the number of calculational cycles in the computer model and correlate directly with time since the initial stimulation of the left ventricle.

An interesting consequence of PRT is to produce a shorter QRS complex than any current pacing modality. This is due to improving activation synchronicity over the entire heart, not just the left ventricle. However, it should again be emphasized that narrowing the QRS complex is not the goal of PRT, rather it is a mere side effect, and that mere narrowing of the QRS has not been shown to correlate clinically with improved cardiac function. PRT also produces good RV intraventricular synchrony. This is important since in the act of improving LV intraventricular synchrony, we do not wish to induce RV heart failure. In the same way, PRT may be of direct value in treating RV heart failure.

Delivering PRT involves stimulating the right ventricular septum simultaneously from roughly the apex to the outflow tract. Since simultaneous activation of the RV septum is desired, this therapy may be deliverable with a standard pacemaker. The right-ventricular lead or leads will connect to the ventricular channel. Standard connectors or specialty connector may be used. However, conventional lead technology will not be adequate for delivering PRT.

The PRT method is not dependent on any specific method for delivering stimulating pulses to the RV septum or on specific ways of constructing leads or electrodes. The electrodes could be wound coils or more conventional ring electrodes or other configurations. Any anchoring technique can be used so long as a sufficient number of electrodes contact the septal wall (it is not necessary that all electrodes contact the wall so long as a sufficient number do and that they more or less evenly stimulate the RV septum). It is also not required that the conductors be insulated from each other since PRT involves stimulating all electrodes substantially simultaneously. However, different electrodes might also stimulate the right ventricular septal wall at slightly different times, in which case insulated conductors would be important. The object is to produce a uniform emerging wave front along the left ventricular septal wall. Because of differing placement of electrodes in the right ventricle and because of physiologic factors such as varying septal wall thickness, stimulation in the right ventricle at slightly different times may be needed to produce the desired effect in the left ventricle. The electrodes should have a small surface area, for example between 1 mm² and 9 mm² when used with an approximately 1 mm diameter pacing lead. The small electrodes will generate lower pacing thresholds and will minimize battery drain when placed in parallel.

The right-ventricular septum is approximately 5 or 6 cm long in adult humans. Sufficient electrodes are required along the RV septum so that when the action potential from each electrode reaches the LV side of the septum, the individual waves will have fused within some time, generally 1 to 4 ms (total fusion is not absolutely necessary, but it is desirable). Because the action potential conduction velocity through the septum is smaller than parallel to it, the action potential generated by each electrode will spread in an ellipsoid through the septal myocardium with the longer semi-major axis parallel to the septum and the shorter semi-minor axis transverse to the septum. Geometrical analysis indicates that five electrodes, all in contact with the septum and spaced approximately 1.5 cm apart, are sufficient to produce a fused action potential on the left ventricular side of the septum in an average adult human within 4 ms of delivering simultaneous pacing pulses with these electrodes. The most proximal electrode is assumed to be located in or near the right-ventricular apex. The most distal electrode is assumed to be located somewhere near the right-ventricular outflow tract, generally somewhere near the moderator band.

More than five electrodes will generate a fused action potential on the LV septum in less time. A preferred embodiment is therefore 9 or 10 electrodes spaced 6 or 7 mm apart. This allows for the possibility that some of the electrodes may not be in good contact with the myocardium. PRT will be delivered so long as at least four or five of these electrodes, spread approximately evenly apart, are in contact with the septum. Sufficiency of contact may be determined in several ways, principally by observing the response of the patient and the patient's heart to stimulation. For example, patients with left bundle branch block (“LBBB”) exhibit a specific ECG pattern. In particular, a QRS of 120 ms or greater, broad monophasic R wave in on standard ECG leads I, V₅, and V₆; absence of Q waves in leads I, V₅, and V₆; delay in the onset of the intrinsicoid deflection in leads V₅, and V₆; and displacement of the ST segment and T wave in a direction opposite to the major deflection of the QRS complex. Successful delivery of PRT will generate an ECG pattern closer to normal (shorter QRS, less broad R wave, more normal displacement of the ST segment and T wave, etc. . . . ). Observation of the ECG with and without PRT will show that the ECG pattern is more normal and that PRT is being delivered.

Furthermore, paradoxical septal wall motion (for example, measured non-invasively with echocardiography) should be reduced. Mitral regurgitation (for example, measured non-invasively with echocardiography) should be reduced. An EP map can be made using a conventional one dimensional or an advanced three dimensional mapping system to verify capture at or near each electrode. A physician might also observe an increase in pulse pressure, cardiac output, and other physiological metrics that correlate with improved LV function. Any of the foregoing may be used to determine that proper physiologic placement of the multi-electrode lead or of multiple electrodes has been achieved.

Preferably, the wave front generated from all the electrodes on the right septal wall will emerge on the left septal wall within 10 ms or less from the time that a wave front first emerges at the left ventricular wall surface, more preferably 2 ms or less. Each of the electrodes in said set of electrodes may produce a stimulating pulse within a period of 2 ms. Also, the electrodes may be connected in parallel with each other. Preferably, at least five electrodes are implanted along the right ventricular septal wall, but the set of electrodes may comprise between eight and sixteen electrodes, or more. The electrodes produce a fused line of stimulation extends from about the left ventricular apex to about the mitral valve and the left ventricular wall surface. The electrodes may be separated from each other by about 7 mm or less and each of the electrodes may have an impedance of about 500 ohms or greater.

In the treatment of congestive heart failure, it may be advantageous to stimulate the right ventricle slightly ahead of an expected physiologic contraction, thereby controlling the contraction of the heart from the RV pacing sites. Thus modern conventional pacers may sense cardiac events in the atrium and wait for a corresponding propagation of the event into the ventricle, stimulating the heart only if timely propagation fails to occur. To treat congestive heart failure, the cardiac stimulator may sense in the atrium, but stimulate in the ventricle at a time sooner than the expected propagation of the intrinsic wave into the ventricles. Alternatively, the ventricle may be stimulated at a rate slightly faster than the patient's expected heart rate. The expected heart rate may be estimated, may be determined by electrical sensing or may be determined by rate responsive sensing, such as by sensing an accelerometer. This anticipatory stimulation allows the cardiac stimulator 12 to control not only the timing of contraction, but also the shape of the contraction, including the preparatory stiffening of the septal wall or muscles around the mitral valve. The shaped contraction is believed to improve cardiac output, and in particular, to allow improved left ventricular performance from electrodes implanted in the right ventricle.

Numerous other modifications may be made to this invention without departing from its scope as defined in the attached claims.

1. A method for stimulating the heart comprising
placing a plurality of electrodes along the right ventricular septal wall surface of a heart, such that stimulation wave fronts from said selected electrodes will stimulate substantially all of the left ventricular septal wall surface substantially simultaneously and
producing a stimulating pulse at each of said electrodes in said set of electrodes at substantially the same time to produce a fused line of stimulation along the left ventricular septal wall surface.

2. The method of claim 1 wherein the step of placing said set of electrodes further comprises generating an elliptical wave front from each of said electrodes such that areas are formed on the left ventricular wall surface where said wave fronts emerge during a pre-selected time and selecting electrodes such that adjacent areas of said areas impinge on each other.

3. The method of claim 2 wherein said pre-selected time is less than or equal to about 10 ms from the time that a wave front first emerges at the left ventricular wall surface.

4. The method of claim 3 wherein said pre-selected time is less than or equal to about 2 ms from the time that a wave front first emerges at the left ventricular wall surface.

5. The method of claim 1 wherein each of said electrodes in said set of electrodes produces a stimulating pulse within a period of 2 ms.

6. The method of claim 5 wherein said electrodes in said set of electrodes are connected in parallel with each other.

7. The method of claim 1 wherein placing said plurality of electrodes comprises implanting at least five electrodes along said right ventricular septal wall.

8. The method of claim 7 wherein the set of selected electrodes comprises between eight and sixteen electrodes.

9. The method of claim 8 wherein said fused line of stimulation extends from about the left ventricular apex to about the mitral valve and the left ventricular wall surface along said line is stimulated with a period of 2 ms from the time that a wave front first emerges at the left ventricular wall surface.

10. The method of claim 1 wherein said left ventricular wall surface along said fused line of stimulation is stimulated within a period of 2 ms from the time that a wave front first emerges at the left ventricular wall surface.

11. The method of claim 1 wherein the electrodes are separated from each other by about 7 mm or less.

11. The method of claim 1 wherein each of the electrodes has an impedance of about 500 ohms or greater.

12. The method of claim 1 further comprising sensing the electrical condition of said heart at least one sensing electrode.
